(19) United States
(12) Patent Application Publication
(10) Pub. No.: US 2025/0204989 A1
(43) Pub. Date: Jun. 26, 2025

(54) METHOD AND SYSTEM FOR PATIENT-SPECIFIC MODELING OF RENAL AND RETROPERITONEAL ANATOMY FOR THERAPEUTIC EVALUATION, PLANNING, CONFIRMATION AND NAVIGATION

(71) Applicant: Jason B. Wynberg, Jersey City, NJ (US)

(72) Inventor: Jason B. Wynberg, Jersey City, NJ (US)

(21) Appl. No.: 18/973,072

(22) Filed: Dec. 8, 2024

Related U.S. Application Data
(60) Provisional application No. 63/696,406, filed on Sep. 19, 2024.
(60) Provisional application No. 63/705,797, filed on Oct. 10, 2024.
(60) Provisional application No. 63/724,628, filed on Nov. 25, 2024.
(60) Provisional application No. 63/614,892, filed on Dec. 26, 2023.

(51) Int. Cl. A61B 34/20; A61B 34/10; A61B 34/30; G06T 5/50; G06T 7/00; G06T 7/10; G16H 30/40

(52) U.S. Cl. CPC A61B 34/20; A61B 34/10; A61B 34/30; G06T 5/50; G06T 7/0012; G06T 7/10; G16H 30/40; A61B 2034/105; A61B 2034/107; A61B 2034/2065; A61B 2034/2068; G06T 2207/20221; G06T 2207/30084

(57) ABSTRACT

An anatomy-based system for presenting and processing patient-specific anatomy to evaluate fitness for surgery, surgical planning, tract confirmation and navigational aid during surgery in the setting of renal nephrostomy creation. A safe puncture path between kidney and flank skin considers the position of any of several organs or anatomic parts in the region of the potential puncture path. The system may include at least one computer system configured to receive patient-specific data regarding a geometry of an anatomic field of the patient including a multitude of anatomic parts and their anatomic relationships to each other. The system may generate three-dimensional modeling of patient-specific anatomy and best planning for a surgical procedure, for example a safe puncture path between the kidney and flank skin. A computer system may be created to represent this anatomic model to enhance understanding of renal puncture safety, planning, confirmation of created tract and live surgical navigation.

### BACKGROUND

Creating a nephrostomy tract involves advancement of a needle between the kidney and the flank skin. To plan the procedure and understand the three-dimensional anatomic field of the retroperitoneal space and patient-specific organ position, the physician reviews available imaging to understand the patient-specific anatomy in the context of the planned procedure. Most imaging reviewed by physicians is presented in a 2-dimensional format e.g. computed tomography, fluoroscopic images, ultrasound images. To best understand the proposed procedure before and possibly during the procedure, a three-dimensional representation of the anatomy is useful as it better represents the three-dimensional nature of the procedure and thus can improve physician understanding and decision making. Three-dimensional rendering can be viewed either on a two-dimensional screen or in a virtual reality 3-dimensional environment.

Various tools exist that categorize patient-specific anatomic factors into complexity levels of kidney stone surgery generally and percutaneous nephrolithotomy (PCNL) specifically. The Guy's stone score, the CROES nomogram, the S.T.O.N.E. nephrolithotomy score and the S-ReSC system are predominately based on kidney stone factors—stone location, stone size, etc. Stone-to-skin distance measurements. In the setting of nephrostomy renal puncture, a sharp member is advanced between the kidney and the flank skin through the retroperitoneal anatomic field with care taken to avoid injuring nearby precious organs. Defining a window of safe passage through the retroperitoneum would be a benefit as it would allow enhanced understanding and potentially measurements. This window of safe passage is herein called a ‘flank window’. Understanding how potential or real renal puncture pathways would pass from kidney to flank skin through the ‘flank window’ would be a benefit to the physician both for procedure understanding, qualification and planning. This ‘flank window’ or safe passage from kidney to flank skin can be presented on 2D or 3D medical imaging.

There is currently a lacking of a system that evaluates this critical soft-tissue anatomy (e.g. nearby organs that are outside the kidney potentially in the path between the kidney and flank skin) to aid in the performance of a nephrostomy creation for the PCNL procedure. Providing a system that focuses on anatomic consideration of non-renal organs in the anatomic field between kidney and flank skin will provide understanding of the anatomic field before and during renal procedures e.g. in the setting of nephrostomy creation. A model that presents patient-specific anatomic detail to the surgeon both before and during the procedure would be a valuable resource to surgeons performing nephrostomy creation and would enhance patient safety and physician adoption of this difficult procedure. Further, a model that adds a proposed or created nephrostomy tract to the patient-specific anatomic field may enhance physician understanding and thus patient safety and adoption of the technique.

Many computer software imaging platforms are currently available to perform anatomic image segmentation and three-dimensional image reconstruction to represent image features relevant to a range of clinical interests. There is an acute need to represent the anatomy related to renal nephrostomy puncture in a three-dimensional fashion. The correlation of established nephrostomy puncture pathways in the intraoperative setting to three-dimensional reconstructed regional anatomy is a function that is not currently available. This requires a system that properly emphases relevant anatomy and de-emphases non-relevant anatomy in the visual presentation of the anatomic field. To enhance that functionality, muscle, fat, bone and skin may need to be segmented, along with kidney, liver, bowel, spleen and pleura, and there may need to be translucency applied to much of these structures to permit ‘through and through’ view of the puncture tract. Collectively, this nephrostomy creation procedure-focused image reconstruction function is not currently reported. An additional value would be to represent on reconstructed imaging a puncture path in relation to the anatomic field. With this function the surgeon can cognitively map the puncture planned or created to the nearby intracorporeal anatomy, or else computer software functionality can map the puncture path onto the image reconstruction data set.

Rules that govern puncture or other surgical interventions such as retrograde nephrostomy wire puncture or tissue biopsy can be defined and applied to this anatomic data to provide puncture tract evaluation, interventional analysis, planning, qualification, and intraoperative procedure confirmation to the physician. Further, the combination of ‘flank window’ anatomic field data with procedural rule sets can be used to assess surgical risk, feasibility, planning, and navigational support during the procedure.

Thus, the present invention endeavors to:
1. Define a ‘flank window’ of safe passage for procedural instrument advancement between kidney and flank skin in consideration of the avoidance of precious organs in the region of the path between kidney and flank skin;
2. Represent relevant and precious anatomic structures possibly including the ‘flank window’ in a fashion to enhance visual understanding of the relevant anatomic field in the context of a planned renal puncture procedure. Representation of modified anatomic image data can be presented to the physician on a flat 2-dimensional screen, 3D printed or with a virtual reality three-dimensional environment to aid understanding of anatomy. This visual representation can aid preoperative procedure evaluation and planning;
3. Enable reconciliation of proposed or performed renal puncture with the preoperatively obtained 3D image reconstructions in the preoperative or intraoperative setting to demonstrate to the physician relative position of the entirety of the renal puncture pathway to relevant nearby organs. This reconciliation can be performed cognitively or using a computer function to represent the puncture path onto the preoperatively obtained 3D image reconstruction set. When the puncture tract is mapped on the 3D image reconstructions, rule sets may permit evaluation of the tract features, measurements, distance to organs, safety, etc.
4. Define and apply rule sets to CT scan ‘flank window’ anatomic reconstruction to present to the surgeon potential puncture paths between kidney and flank skin. These rule sets can provide procedure modeling in the preoperative setting.
5. Other procedural navigation elements such as video endoscopic image registration and tracking to these processed and reconstructed images to be presented in an image fusion and/or augmented reality functionality in the live procedure setting.

### SUMMARY OF INVENTION

In an exemplary embodiment, a model or system that defines an anatomic window (‘flank window’) from the kidney, through the retroperitoneum, to the nearby ipsilateral flank skin. This window is defined either by its nearby precious perimeter organs where the space between these precious organs is the safe anatomic ‘flank window’ or by a manually drawn field drawn inside of the space defined by the perimeter organs where the perimeter of this drawn field may be a distance from the perimeter anatomical organs. This window can be represented visually in various fashions to a physician to aid in understanding the patient-specific anatomy relevant to a procedure.

In an exemplary embodiment the ‘flank window’ may be defined in several ways:
A) Considering some or all of the following anatomic aspects of nearby organs: medially by the lateral edge of paraspinal muscle; anteriorly/laterally by the nearest viscera—e.g. liver, spleen, colon, pleura; superiorly by spleen, liver, pleura or 11th rib; inferiorly by colon or anterior superior iliac spine;
B) Manual segmentation, rule-based automatic or AI-supported segmentation process applied to CT or MRI scan image data wherein the flank window perimeter may be set a distance inside the inner boundary of perimeter precious organs. By making the flank window a distance from perimeter organs, a margin of safety to avoid contacting the precious perimeter organ is provided to a planned procedure. This segmentation of the flank window may be drawn onto images manually using a drawing tool or AI and/or rule-based automated process and these images can be reconstructed into a three-dimensional model.

In an exemplary embodiment, a 3D imaging reconstruction of previously acquired source patient-specific imaging data demonstrating the kidney and other structures in the region of the path between the kidney and the flank skin possibly including perirenal and subcutaneous fat, the position and terminal points of one or more ipsilateral ribs, colon, pleura, liver, spleen, paraspinal muscle, pararenal and perirenal fat, fascia, flank musculature, subcutaneous fat, possibly with specific visual representation of the ‘flank window’ which defines a safe path between kidney to skin. Renal specific anatomic features may be incorporated to enhance position and structure information e.g. renal rotation, anterior vs. posterior regions of kidney, upper/mid/lower pole regions of kidney, renal infundibulae, renal papillae, subsegments of renal papillae, any renal masses, etc. Renal anatomic feature input may be limited by imaging detail availability e.g. CT urography and or retrograde pyelography may provide more anatomic information for segmentation than a source CT that was acquired without contrast. All of the above anatomic reference points may be segmented using a combination of manual and automatic or AI supported processes. This visual image reconstruction will present selected anatomic elements onto a single visual spatial model. This unified multidimensional visual model may aid the surgeon in planning an antegrade or retrograde renal procedure by presented a clear view of the relevant anatomy. In an exemplary embodiment this model would be applied to either antegrade or retrograde renal puncture for nephrostomy creation. Translucency may be applied to the 3D image reconstruction to various anatomic elements to permit enhanced visual understanding of the path from kidney to flank skin while still understanding the relevant regional anatomic structures and their interrelationships to each other and a potential safe puncture path between kidney and skin. This 3D reconstruction can also be formatted and processed to permit ‘drive through’ or ‘virtual puncture’ journey in 3D space between kidney and flank skin using a virtual reality or alternative software feature. Puncture path may be modeled if the puncture point on each of the kidney and flank skin are selected, with the shortest line between them becoming a model puncture path through the anatomic field. The kidney puncture point is cognitively known to the surgeon as they have awareness that the puncture was through a specific region of the kidney e.g. posterior kidney and mid pole, for example. Thus, with the skin puncture site known in relation to, say the tip of the 12th rib, and the renal puncture site being known, review of the three-dimensional regional anatomy with the puncture beginning and end points known, the tract can be overlaid onto that pre-existing reconstructed 2D or 3D image set either cognitively (in the ‘mind's eye’ of the surgeon) or can be actually visually overlaid onto the 2D or 3D reconstructed images using computer functionality.

In an exemplary embodiment, the above anatomic elements are represented as voxel coordinates in a single unified spatial model (X-Y-Z planes) to allow accurate visual image presentation of relative organ/anatomy position, and also to permit procedure modeling on top of this 3D anatomic field converted to specific multiplanar position data.

In an exemplary embodiment, information regarding a proposed or intraoperatively performed renal puncture can be added to the above XYZ anatomic positional data to model to visually model a puncture path relationship to relevant nearby anatomic structures. Anatomic structures that permit registration of the actual puncture to image reconstruction may include palpable ribs or portions thereof, skin overlying the palpable rib, the posterior axillary line, the umbilicus, the palpable lateral edge of the paraspinal muscle, skin, subcutaneous fat, fascia and flank musculature, pararenal and perirenal fat, and possibly a radio-visible marker that may have been applied to the patient prior to acquisition of source CT scan imaging.

In an exemplary embodiment, a model or system defines and applies interactive rule sets to a spatial model that evaluates and proposes potential puncture paths between kidney and flank skin while passing through the ‘flank window’. Input function may be added to allow the physician to interactively contribute to selected puncture path. This modeling may inform the surgeon who is planning an antegrade or retrograde renal procedure, such as nephrostomy creation, as to available and optimal paths. In one exemplary embodiment this model would be applied to retrograde renal puncture for nephrostomy creation. In one exemplary embodiment the proposed or best selected path rule set would have additional outputs such as distance to nearest precious organ, categorization into ‘safe’ and ‘unsafe’ groupings, etc.

In an exemplary embodiment, the above model or system may use various methods to propose a puncture path from the renal collecting system to the flank skin passing through a safe anatomic window (‘flank window’). These puncture path models can be interactive with the physician, permitting the surgeon to modify and manipulate the puncture paths for example with a drawing tool. Methods to determine a puncture path include but are not limited to: (i) AI, neural networks or other advanced calculation methodologies can be applied to select a ‘best path’ between the kidney and the flank skin while passing through the flank window, where ‘best path’ may consider any of: angle of approach limits to the skin, preferring to be closer to the center of the ‘flank window’ or at least a distance from the edge of the ‘flank window’ or nearby organs, and any preference regarding approach to or departure from the kidney. Regarding these points, physician input may be used to inform the ‘best path’ calculation e.g. ‘no puncture from or to the upper pole’ or ‘no puncture from or to the anterior region of kidney’ or ‘path must be from or to a physician selected calyx/infundibulum’ or ‘no puncture path angle of approach to skin of greater than 15 degrees from perpendicular’, etc. Specific regions within a calyx e.g. posterior fornix can be calculated as part of a ‘best path’ output; (ii) a retrograde puncture path is established by selecting a minimum of 2 points in X-Y-Z space from within the renal collecting system from which the retrograde puncture path is established. These initiation points can be proposed by AI or other advanced calculation systems or by physician using a manual process e.g. manual segmentation. In either case, the puncture path trajectory is linearly extrapolated from the path set by these initiation coordinates in X-Y-Z space. A margin of error around the ideal puncture path will predict a potential range of puncture paths and this range of error will grow over distance in space as the wire progresses further from the initiation of the puncture. This consideration may or may not be applied as a rule set to build a confidence level of possible wire paths in space; (iii) two or great point position data on the ureteroscope tip is used: e.g. where 2-point position of the ureteroscope tip is captured either by fluoroscopic registration where CT/fluoroscopy fusion is available, or by visual registration; (iv) a range of positions within an infundibulum and calyx are calculated to determine the optimal position on the papilla for puncture. In an exemplary model a specific rule set considers the angle of approach of the puncture wire to the flank skin. A more tangential approach to the skin creates a weaker puncture interface of the wire with the skin which can reduce power of the puncture wire to advance through the skin. Thus, rules may place a limit on ranges of puncture angles to ensure an acceptable wire angle at the flank skin.

The above outputs can be presented as sequential 2D anatomic slices or as a 3D reconstructed model. The 3D images can be presented on a two-dimensional screen or else presented in three-dimensions e.g. as a 3D printed model, in a virtual ureteroscopy or virtual reality environment. This representation provides the surgeon spatial understanding of the patient-specific flank anatomy and patient-specific safe anatomic windows for puncture from the kidney to the flank and can be used by the physician to assess surgical candidacy, surgical planning and risk. This can be applied in the pre-operative planning stage or intraoperatively as a cognitive aid to the surgery.

In an exemplary embodiment, a system or model wherein a therapeutic renal procedure plan can be assessed and created based on rule set modeling and case qualification and these outputs can be presented to the surgeon on a 2D monitor or 3D printed model or virtual ureteroscopy or virtual reality environment, and these data outputs may allow the surgeon to interact with them either in a preoperative or intraoperative environment.

In an exemplary embodiment, computer-based method for segmenting relevant anatomy, registration with live endoscopy and tracking e.g. image based, optical or electromagnetic system to provide live navigational assistance during the surgery by overlaying preoperative computational modeling for safe, ideal, and/or preselected puncture pathways with live surgical position. In an exemplary embodiment this model may comprise: receiving processed image data of a patient's abdominal and retroperitoneal anatomy and ‘window of safety’; applying puncture rule sets to processed anatomic data to determine best or possible puncture initiation points and best or possible puncture angles; receiving live endoscopic image data of a portion of the patients renal collecting system collected from a flexible ureteroscope and possibly fluoroscopy image or ultrasound data; performing image registration between these image data to determine a global location and orientation of the ureteroscope within the patients urinary collecting system during flexible ureteroscopy and provide guidance on how to perform the selected procedure according to the previously modeled and approved puncture trajectories determined by pre-operative image analysis and puncture path rule set application. Image tracking, optical tracking, electromagnetic tracking methods can be applied to the registration system to provide live ongoing procedural guidance.

Create a rule set to determine overall case complexity—‘easy’; ‘moderate’; ‘advanced’; ‘unsafe’. This can be based on a number of considerations some or all of which may include distance and angle calculations, safe anatomic window size, distance from kidney to skin, etc.

In an exemplary embodiment:

Cross-sectional imaging data is sent to a software program to segment relevant abdominal and retroperitoneal anatomic structures, ribs, detailed anatomic features of the kidney, flank skin, flank muscles, fascia, pararenal and perirenal fat, subcutaneous fat and possibly the ‘flank window’. This segmentation process may incorporate automatic, manual and/or AI supported segmentation processes.

Render a visual reconstruction of the above and present this as a 2D slice-by-slice reconstruction or a 3D reconstruction to the surgeon either on a 2-dimensional monitor, a 3D printed model, or in a 3D virtual ureteroscopy or virtual reality environment. This output can be presented to the physician in the preoperative environment or intraoperatively as an aid to surgery.

In another exemplary embodiment, the above outputs 1. and 2. can be used as source processed imaging data and the following functions are added on top of that existing output:

In the preoperative or intraoperative setting of an antegrade or retrograde nephrostomy puncture, anatomic points such as (i) the skin puncture possibly in positional relation to nearby identifiable bony anatomy e.g tip of 12^{th} rib, and (ii) renal puncture points, can be used as the two ends of an interpolated line between them and this interpolated line can be cognitively modeled by the surgeon to consider the puncture path or else are inputted onto the 2D or reconstructed 3D anatomic image data set using rule sets and tools such as a manual drawing tool or description-based input into a computer system able to receive and model or interpolate a straight line between said anatomic points, such as (i) the skin and possibly its positional relation to bony landmarks e.g. 12^{th} rib tip, and (ii) renal puncture points and model these points and a line between them onto the preoperatively 2D slice-by-slice or 3D reconstructed anatomic images. This would permit the physician to view a modeled or intraoperatively actual puncture path's relationship to relevant nearby organ structures. For example, after a surgeon creates a puncture intraoperatively, the wire emerges from the flank skin and is controlled with a clamp. The surgeon measures the distance from the nearest palpable rib or other known and segmented anatomic structure and this information becomes the skin puncture data point. The surgeon is aware from where in the kidney the puncture penetrated the kidney. With these two points (skin and kidney) known in XYZ planes, the puncture path through the flank tissues can be interpolated between these two points either in a cognitive ‘mind's eye’ fashion of a physician, or a function can exist to permit entry into a computer system of these puncture data at skin and kidney, and the computer function will model the created puncture path between these two points and present this line on either 2D slice-by-slice or 3D reconstructed images, permitting the surgeon to evaluate their created tract and the path's relation and proximity to nearby organs, angles, etc. Additionally, further rule sets can provide additional outputs relevant to evaluation of the created puncture tract such as distance from any point along the puncture path to a nearest precious organ, overall safety profile based on established rule set criteria, total tract length calculations, angles, etc. Maximum and minimum values can be included in these rule sets. The entry (antegrade puncture) or exit point (retrograde puncture) of the wire in relation to the skin could be registered to either the 2D axial, coronal or sagittal images or the 3D segmented and reconstructed images using an anatomic structure common to both the external flank anatomy and the 3D images such as a portion or point on a specific palpable bony structure e.g. rib or the skin overlying the palpable bony structure e.g. rib, and the relation of the wire skin entry or exit to that identified portion of bony structure e.g. rib and its corresponding representation on the 2D or 3D segmented preoperative images, or else other structures such as umbilicus, posterior axillary line, edge of paraspinal muscle, radio-opaque marker, etc. A 2D slice-by-slice or a 3D representation can be generated that shows the puncture path (straight line drawn in 3D between skin and renal wire puncture points) in the larger anatomic field, to determine puncture safety prior to proceeding further in the operating room with the subsequent steps of the procedure. Regarding the renal entry (in the setting of an antegrade nephrostomy puncture) or exit point (in the setting of a retrograde nephrostomy puncture), information such as anterior or posterior, upper, mid or lower pole regions of the kidney may be used to register or model the true puncture to the XYZ spatial data grid and reconstructed images.

In another exemplary embodiment, the above outputs 1. and 2. (and possibly 3.) can be used as source processed imaging data and the following functions are added on top of that existing output:

During the procedure planning stage, apply a set of rules that assess potential puncture paths initiated from various positions from within the kidney. These sets of rules include but are not limited to sub-rule sets that (i) define the origin of the puncture path from within the kidney; (ii) constrain puncture paths to safely travel through the segmented ‘window of safe passage’ based on maximum distance tolerable to nearby organs, etc.; (iii) constrain acceptable angles and lengths of puncture wire to contact flank skin; (iv) maximum tract length. Alternatively inputted renal and skin puncture site data (3. Above) can be analyzed against rule sets to evaluate the safety of the entered tract data.

Output of this phase may include (i) treatment tract ratings/proposals, including which segment of papilla is ideal for puncture to achieve proper path angle to treatment target area e.g. puncture through ‘window’; (ii) simulated 2D fluoroscopy image showing range of acceptable flexible ureteroscope positions in 2D space to achieve acceptable wire angles; (iii) overall complexity score of the case—‘easy’; ‘moderate’; ‘advanced’; ‘unsafe’; (iv) video reconstruction of urinary collecting system with eligible puncture paths visually overlaid onto this to simulate the surgery plan possibly with a virtual endoscopy or virtual ureteroscopy element to the presentation of these reconstructions. This phase aids with preoperative surgical decision making and planning.

In another exemplary embodiment, the above outputs can be used as source data and the following functions are added on top of that existing output:

A possible extension of the above output is registration and tracking of live video endoscopy (possibly also live fluoroscopy and/or ultrasound images) with preoperatively processed images is performed (1) using soft tissue urinary system targets that can be identified on both 3D CT and vision endoscopy; (2) registration may be integrated with fluoroscopy (biplanar or multiplanar) and/or ultrasound. Image registration is performed by any or more of (1) marking the ureteroscope position (i) under vision and optionally (ii) in relation to live retrograde pyelogram to the corresponding 3D processed CT image, optionally with integrated CT urogram or retrograde pyelogram data. Tracking can be performed using image-based tracking system, optical tracking or electromagnetic tracking.

In an exemplary embodiment, soft tissue registration targets can include the ureteropelvic junction (UPJ), any number of calyces (anterior/posterior calyces, uppermost, lowermost calyces, the ostium of the selected infundibulum and the papilla of that infundibulum selected for puncture.

In an exemplary embodiment, registration may include various points on a particular papilla—cephalad fornix, posterior fornix, etc. In particular, soft tissue registration data from the infundibular ostium and papilla selected for puncture can be abstracted in XYZ planes and used to generate the origination point and path prediction for the puncture path probability cone.

In an exemplary embodiment, continuous and discrete ongoing/subsequent registrations can be performed based on visual travel through the mapped branched lumen of the kidney and/or known anatomic landmarks.

In an exemplary embodiment, registration and/or tracking can be performed using image-based tracking systems, optical tracking, EM navigation or other identifiable landmarks on fluoroscopy such as ribs, the flexible ureteroscope itself, vertebrae, anterior superior iliac spine, etc. These data may be integrated with direction vision endoscopic/preop imaging data registrations and/or tracking systems.

In an exemplary embodiment, registration and tracking can additionally be integrated with 2D fluoroscopy if CT/fluoroscopy fusion capabilities are available/integrated, ultrasound or electromagnetic (EM) navigation where a 2-point EM probe is inside the working channel or integrated into the distal 2 cm of the flexible ureteroscope. In the scenario of 2-point EM based inside the ureteroscope either as a built-in feature or a probe in the ureteroscope working channel, having 2-point data spatial data inside the kidney permits an accurate extrapolation of a retrograde advanced puncture wire.

In the intraoperative environment, several outputs are contemplated: 1. integrated visual of live endoscopy video and color-coded guidance overlay onto the endoscopic video, to guide the physician to the preselected region (Upper/mid/lower pole, anterior/posterior) or specific calyx for puncture as well as sub-portion of papilla to aim for (e.g. ‘aim for posterior portion of papilla’); 2. 3D representation of patient-specific anatomy and flank window for surgeon review alongside the surgery, possibly with preoperatively proposed or selected puncture paths; 3. Two-dimensional representative image of ureteroscope biplanar position on simulated fluoroscopy to define safe range of ureteroscope position for puncture—e.g. 0 degrees cephalad/caudad down to 20 degrees cephalad caudad, to guide physician to set safe ureteroscope position on 2D fluoroscopy; 4. Projection of soft tissue anatomy or approved puncture pathway data onto 2D fluoroscopy images; 5. Augmented reality display.

In an exemplary embodiment of this invention the entirety of the systems defined herein are applied to permit various instruments to be extended from the ureteroscope working channel under the entire range of the imaging and procedural concepts and guidance systems defined herein to diagnose or treat other issues e.g. direct a biopsy instrument to biopsy a segmented renal mass, therapeutic needle either cycling cold liquid for cryotherapy or a needle with a lumen to inject a therapeutic or diagnostic agent including but not limited to immunotherapy or chemotherapy; intervene for diagnostic or therapeutic purposes using a ureteroscope to deploy therapy or intervention to the liver, colon, spleen, skin, etc.

The method further comprises fusing the endoscopic image data with the 3D CT scan or MRI image data, possibly with biplanar or multiplanar fluoroscopy data to provide real time navigational assistance to the surgeon during the procedure. The method further comprises projecting navigational guidance onto the live endoscopy image. The method further comprises superimposing the endoscopic position onto a processed 3D reconstructed CT or MRI image. The method further comprises superimposing image data—video or 3D CT or MRI data onto fluoroscopy images.

In an exemplary embodiment of the present invention, a system for flexible ureteroscope navigation assistance, comprising: a memory device for storing a program; a processor in communication with the memory device, the processor operative with the program to: receive first image data of a patients abdominal and retroperitoneal anatomy, the first image data acquired before a surgery is performed; a segmentation process, either automated or manual, to mark relevant anatomic sites on the first image data; a rule set or sets applied to the image data in the program to map potential anatomic pathways through the anatomy; receive second image data acquired during the surgery procedure (endoscopic video and/or fluoroscopy data and/or ultrasound data); and perform registration between the first image data and the second image data to determine a global location and orientation of the ureteroscope within the patients urinary collecting system during the surgery and to inform the operator on how to best proceed with the procedure.

In an exemplary embodiment of the present invention, a system for real-time flexible ureteroscopic procedure planning based on 3D CT or MRI image data (and possibly retrograde pyelographic or ultrasound data) and applied rule sets to these image data. Navigational assistance is provided with various strategies including, but not limited to, fusion of images, separately displayed images, etc. Any combination of image overlay or guidance instructions between 3D CT/MRI data, endoscopic video, fluoroscopy and ultrasound is contemplated—whichever presentation is most useful for the surgeon will be presented.

It is contemplated that the rule sets will require iterative improvement as a multitude of patient-specific data is collected, to improve performance of predictive elements of surgical guidance outputs. Various means of iteration are contemplated which will result in improved algorithm performance.

The tools and sequences described herein can be applied to a range of organ systems for diagnostic and/or therapeutic purposes. One alternate embodiment is for the diagnosis and treatment of renal lesions using a retrograde approach through the collecting system. More generally, other embodiments may include the application of these principles to any endoluminal approach to diagnostic or therapeutic deliveries involving non-renal organs such as sinuses, gastrointestinal system, central nervous system procedures, cardiovascular procedures.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

It should also be understood that the above descriptions and those represented in the drawings are only representative of illustrative embodiments. For the convenience of the reader, these descriptions have focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, are not be considered a disclaimer of those alternate embodiments. Other applications and embodiments can be implemented without departing from the spirit and scope of the present invention.

It is therefore intended that the invention not be limited to the specifically described embodiments because numerous permutations and combinations of the above and implementations involving non-inventive substitutions for the above can be created, but the invention is to be defined in accordance with the general principles that are contained herein. It can be appreciated that many of those undescribed embodiments are within the literal scope of claims to be filed, and that others are equivalent.

### BRIEF DESCRIPTION OF THE DRAWINGS

FIG. 1 illustrates a method for flexible ureteroscope procedure planning and intraoperative navigational assistance based on 3D CT or MRI image data and rule sets applied to 3D image data.

FIG. 2 illustrates a method for procedure planning and real-time ureteroscopic navigation according to an exemplary embodiment of the present invention.

FIG. 3a illustrates the marking and recording of anatomic regions of interest in a renal collecting system.

FIG. 3b illustrates the marking of a ‘flank window’ with precious organs surrounding the ‘flank window’.

FIG. 3c illustrates the marking and recording of anatomic regions of interest in a renal papilla.

FIG. 3d illustrates the marking and recording of anatomic regions of interest on a pyelographic image.

FIG. 3e illustrates the distance from kidney to flank skin.

FIG. 4a demonstrates relevant flank anatomic regions of interest and puncture trajectory modeling mapped onto a single XYZ graph.

FIG. 4b demonstrates line and angles calculations from kidney to skin.

FIG. 5a demonstrates an image fusion between video endoscopy and a procedural guidance rule set output, directing a ureteroscope to a preferred location for intervention while avoiding an area of the kidney disqualified for puncture.

FIG. 5b demonstrates an image fusion output of a procedural guidance rule set directing a puncture path to a specific fornix of the renal papilla onto endoscopic image.

FIG. 5c demonstrates a procedural guidance rule set output of a range of acceptable flexible ureteroscope puncture pathways modeled to a 2D fluoroscopy image.

FIG. 5d demonstrates a rule set output providing risk categorization of a proposed puncture path based on patient-specific anatomy.

FIG. 6 demonstrates a 2D axial CT image with ‘flank window’ segmented, along with segmentation of other anatomic structures.

FIG. 7a demonstrates a 3D reconstructed computed tomography scan showing relevant anatomy.

FIG. 7b demonstrates a 3D reconstructed computed tomography scan showing relevant anatomy and flank window.

FIG. 8 demonstrates a 3D reconstructed computed tomography scan with relevant anatomy and puncture path in the setting of nephrostomy creation with an example of distance measurements to relevant nearby structures represented.

FIG. 9a demonstrates a nephrostomy puncture wire outside the flank with a distance measurement notated in relation to the palpable tip of the 12th rib. This distance measurement to the palpable tip of the 12th rib is entered into a computer as a data point to contribute to modeling of the actual puncture path.

FIG. 9b demonstrates an anatomic site of puncture through a specific subregion of kidney.

FIG. 9c demonstrates an output of a modeled actual nephrostomy puncture path onto the 3D reconstructed images and distance measurement calculations.

FIG. 10 illustrates a method for intraoperatively performed nephrostomy puncture path reconciliation with previously processed 3D reconstructed images.

FIG. 11 illustrates a method for procedure planning and intraoperative image mapping of created nephrostomy tract according to an exemplary embodiment of the present invention.

### DETAILED DESCRIPTION

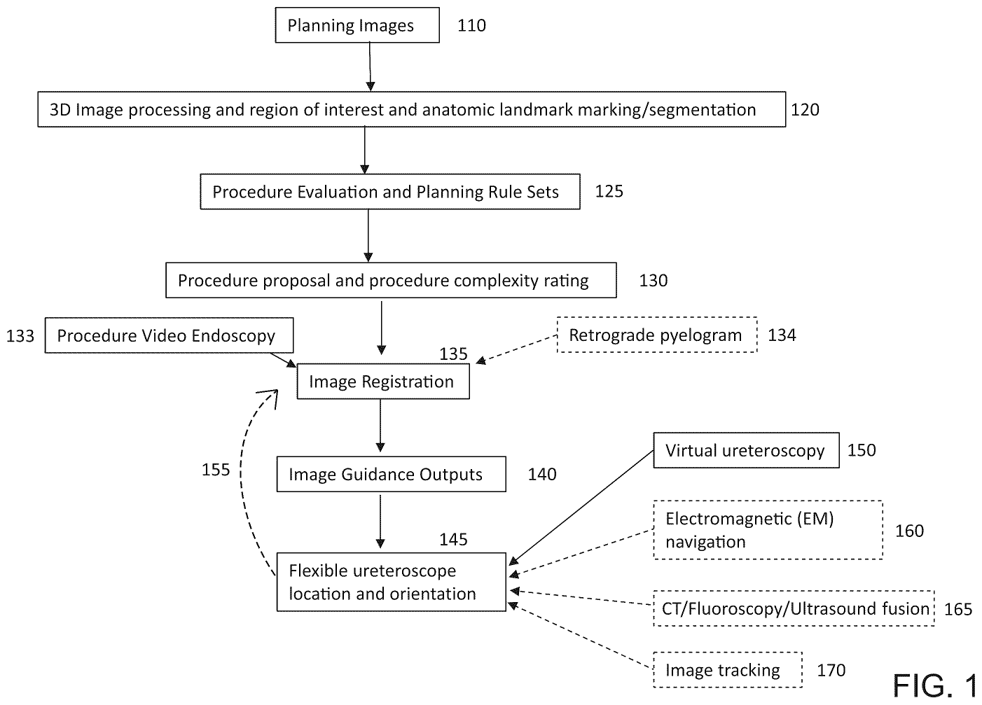

FIG. 1. illustrates a method for flexible ureteroscopic procedure planning and navigational assistance according to an exemplary embodiment of the present invention. As shown in FIG. 1, a planning image or images are acquired from a patient 110. This is done, for example, by scanning the patient using the acquisition device 205 to generate a series of 2D images such as image slices through the abdomen and retroperitoneum, or a retrograde pyelogram. The 2D images are then (i) combined to form a 3D image of the patient's anatomy, and (ii) regions of interest and segmentation are marked 120. These processes (i) and (ii) can be done concurrently or separately, may use artificial intelligence or other software or by manual segmentation of images. For example, the collecting system, papillae, renal contours, liver/bowel/spleen/paraspinal muscle/skin/etc. Anatomy can be marked manually to a receiving computer input with touch screen, computer mouse, or with separate software or applications. This data is stored in the memory 230 and/or viewed on the display 260. Software applications such as “3D Slicer” or DICOM ITK may be utilized here, however, similar functions are available from other commercially available software systems.

Once the 3D image reconstruction and ROI marking and segmentation 120 has been performed, procedure planning and navigational assistance rule sets 125 are applied to this data 120 to generate an output 130 of (i) acceptable treatment plan proposal(s); (ii) and virtual navigation through collecting system imaging to review procedure plan proposal and rating 505; (iii) proposed 2 dimensionally represented anterior/posterior simulated fluoroscopic position of ureteroscope positions for acceptable puncture for each acceptable treatment plan 510. To the extent that rule sets will rule out pathways that do not meet criteria for puncture path eligibility, only those pathways that qualify will be represented as options for puncture. For example, if a puncture from a region of the kidney cannot pass through the safety window based on extrapolation from puncture site initiation 405, then that region of the kidney will be ruled out as having a viable path for puncture. For viable puncture pathways, various modes of visual presentation of treatment plan proposals 130 are contemplated. Various aspects of the treatment proposals can be represented including but not limited to (i) visually represented paths to avoid, and to the papillae suitable for puncture; (ii) sub-regions of selected papilla ideal for puncture (e.g. posterior portion of papilla may be marked for targeting). A rating or case complexity score is also contemplated that would be the product of a rule set that informs the surgeon of the case difficulty or feasibility. A relative complexity scoring of one or more potential procedure paths is contemplated which could result in a ranking of first and second choice pathways, for example.

Given the procedure plan proposal and complexity rating 130, surgeon can review these prior to surgery to qualify or disqualify a patient for surgery, plan the surgery, and review the surgical plan visually prior to surgery or as an aid during the surgery. This output 130 can also be integrated in the OR to provide live surgical guidance. In this embodiment, a video endoscopy image is acquired from the patient 133 using the flexible ureteroscope 270 and fluoroscopic data 134 may be integrated into the registration/tracking system.

Next, image registration is performed between the planning image 120 and the video endoscopic image 133 and possibly fluoroscopy data 134. This is done by performing any number of image registration techniques. These may include identifiable soft tissue anatomic sites on endoscopy and planning image 120, fluoroscopy image fusion with video and/or 3D CT/MRI data, electromagnetic navigation if integrated, or acquisition at this time of retrograde pyelogram 134. Other methods of registration are contemplated. At this time a deformable mapping can be computed.

Several tracking systems are contemplated: Image tracking, optical tracking, EM navigation tracking.

With the image registration complete, a global location and orientation of the flexible ureteroscope 270 within the urinary collecting system is presented as image guidance output 140. For example, given the location of the ureteroscope 270 within the procedural image, the deformable mapping computed above can then be used to find the location of the ureteroscope 270 in the planning image 120. In order to infer the orientation of the ureteroscopy 270 in the planning image 120, an orientation of the bronchoscope 270 must be determined from the procedure image.

Depending on what is required, several options for image guidance outputs 140 exist at this stage. In one option, for example, the procedure proposal image 130 can be superimposed onto the endoscopy video image 133. Alternatively, the video endoscopy image 133 can be superimposed onto the procedure proposal image 130 and/or its pre-selected procedure path. In another option a simulated 2D fluoroscopy image can be presented to show the surgeon acceptable ureteroscope deflection angles on 2D fluoroscopy acquired during the procedure. In another option, a cine loop can be presented to guide the surgeon to the preselected region of the kidney.

As the ureteroscope is advanced 145, virtual ureteroscopy 150 can be applied to guide the ureteroscope to the preselected target. This is performed by the surgeon reviewing preoperatively generated guidance outputs 130 during the operation. Image re-registration 155 can be performed as the ureteroscope moves closer to its target and passes pre-set regions of interest thereby reducing future positional XYZ error. Tracking can be performed in a number of possible ways including but not limited to image-based tracking, electromagnetic navigation 160 and biplanar or multiplanar fluoroscopy image fusion 165.

Image based or other listed tracking systems 170 may employ registration refinement using 3D-3D modified iterative closest point (ICP) registration. Simultaneous localization and mapping (SLAM) algorithmic frameworks may be used for image-based tracking. Because a flexible endoscope does not have the option of direct motion estimation using an external tracker since the endoscope tip position cannot be obtained via rigid transformation, modelling the tip motion using boundary conditions and dynamic constraints, and applying repeated registration to anatomic points further along the anatomic can provide meaningful location information. As well, because the path of the flexible scope is constrained by the lumenal wall which can be constructed from preoperative images, this highly constrained possible scope position enables the scope tip position to be reasonably computed by referencing medical images. Thus, image registration is a potentially good choice.

Cephalad-caudad ureteroscope tip angle data can be obtained from biplanar fluoroscopy and this data can be integrated into the puncture path calculations intraoperatively 165 to the overall algorithms and guidance outputs for the surgeon. Multiplanar fluoroscopic data can also be integrated to provide 3D position data of the ureteroscope to be integrated to the overall algorithms and guidance outputs for the surgeon 165.

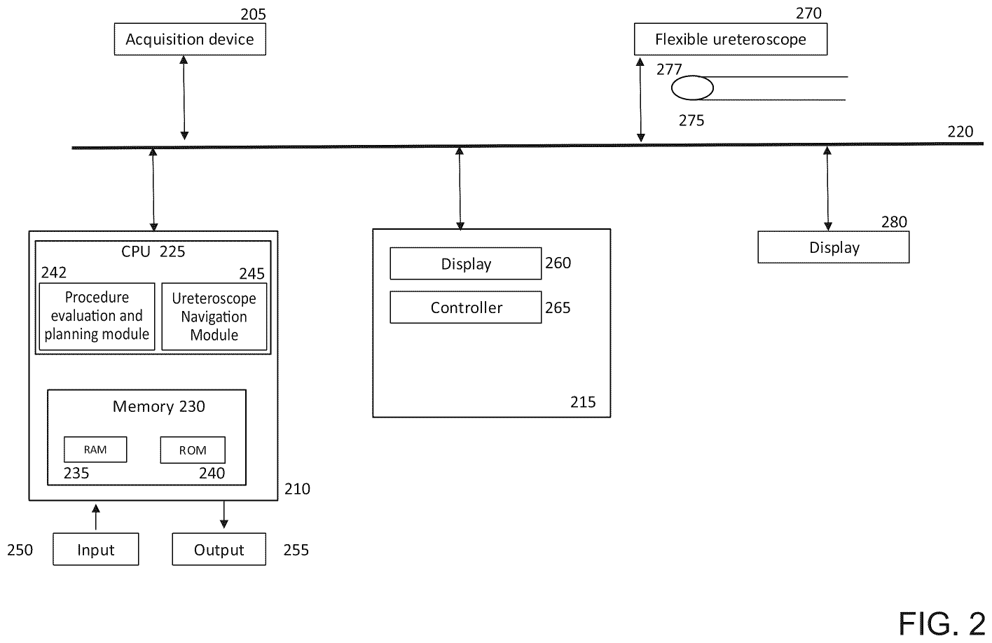

FIG. 2 is a block diagram illustrating a system 200 for flexible ureteroscopic procedure planning and real time ureteroscopic navigational assistance according to an exemplary embodiment of this invention. As shown in FIG. 2, the system 200 includes an acquisition device 205, a computer 210, an operator console 215, a ureteroscope 270, and a display 280 connected over a wired or wireless network 220.

The acquisition device 205 may be a computed tomography (CT) imaging device or any other three-dimensional (3D) high resolution imaging device such as magnetic resonance (MR) scanner. The acquisition device may additionally include a fluoroscopy image acquisition device such as a C-arm or fixed fluoroscopy table or image collected from CT scan scout/planning image which presents 2-dimensional x-ray data.

The computer 210, which may be portable or laptop computer, includes a CPU 225 and a memory 230 connected to an input device 250 and an output device 255. The CPU 225 includes a procedure evaluation and planning module 242 and a flexible ureteroscopic navigation module 245, each of which contain one or more rule sets 125 and methods. Although shown inside the CPU, the planning module 242 and flexible ureteroscopic navigation module 245 can be located outside the CPU 225.

The memory 230 includes a RAM 235 and a ROM 240. The memory 230 can also include a database, disk drive, USB drive, Wi-Fi, Bluetooth, cloud connection, VPN, wired connection or combination thereof. The RAM 235 functions as a data memory that stores data used during execution of a program in the CPU 225 ad is used as a work area. The ROM 240 functions as a program memory for storing a program executed in the CPU 225. The input 250 is constituted by a keyboard, mouse, etc. and the output 255 is constituted by an LCD, CRT display, printer, etc.

The operation of the system 200 can be controlled from the operator's console 215, which includes a controller 265, e.g. a keyboard or touchscreen, and a display 260 which may have an integrated touch screen controller 265. The operators console 215 communicates with the computer 210 and the acquisition device 205 can be rendered by the computer 210 and viewed on the display 260. The computer 210 can be configured to operate and display information provided by the acquisition device 205 absent the operator's console 215, by using e.g. the input 250 and output 255 devices to execute certain tasks performed by the controller 265 and display 260.

The operators console 215 may permit manual anatomic segmentation and may further include any suitable image rendering system/tool/application that can process digital image data, including automatic anatomic segmentation, of an acquired image dataset (or portion thereof) to generate and display images on the display 260. More specifically, the image rendering system may be an application that provides rendering and visualization of medical image data, and which executes on a general purpose or specific computer workstation. The computer 210 can also include the above-mentioned image rendering system/tool/application.

It is considered that some or more of the above functions may be remotely located within the same building or offsite at a remote location with connection via secure data transfer over VPN, cloud, Wi-Fi, Bluetooth, or other modalities. Functions which may be hosted offsite may include the CPU 225 and the memory components 230.

The flexible ureteroscope 270 is a slender tubular instrument with small light 275 on the end for illumination to permit an image capture element 277 to capture and transmit images of the interior of the urinary collecting system for display on a video display monitor 280.

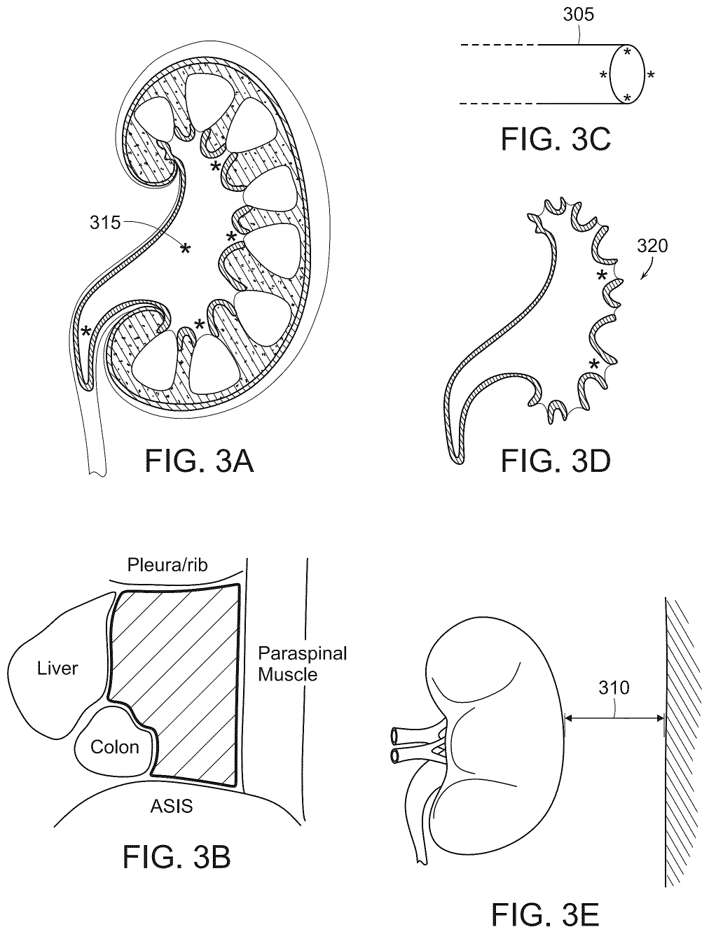

FIG. 3 depicts by asterisk (*) and bold highlighted lines (/) some examples of regions of interest to be marked on 3D image reconstruction 120. These markings are not meant to be the total set of marked points but are merely a sampling of such data. Data from all organs will be segmented or marked either by automated software, mouse clicking or drawing, or touch screen. In addition to marking the edges of relevant structures (e.g. paraspinal muscles, liver, spleen, kidney, colon) to define the safety window 325, 420. Collecting system margins, anterior and posterior calyces 315 and uppermost and most caudad calyces 320 will be marked. Marking may also be performed on various portions of the papillae of interest 305 (e.g. anterior aspect, posterior aspect, cephalad or caudad aspect of papilla). Relevant measurements will also be taken 310. These data points will be mapped onto an XYZ map and will be then subjected to procedure planning rule sets 125. Distance measures will be included in the data set.

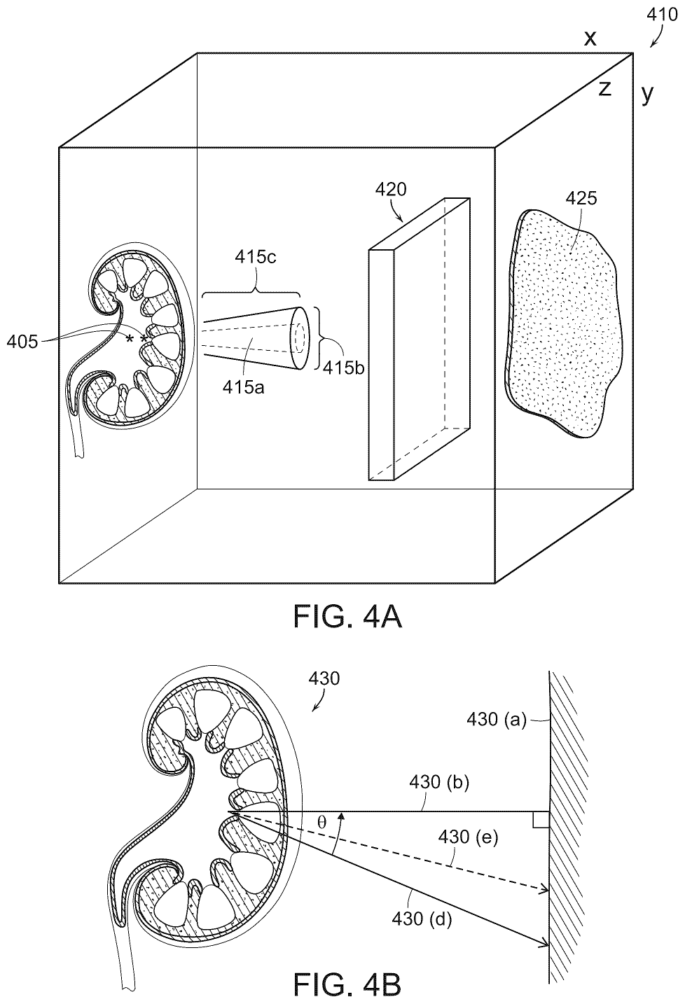

FIG. 4 represents the application of the rule sets to the data. Extrapolation of puncture path from any 2 or more points from within the kidney is considered. Several anatomic points can be used to establish the puncture initiation which is then applied to a rule modeling via extrapolation the likely puncture path from kidney to the flank skin. Image 405 demonstrates a non-limiting example of two anatomic points of infundibular ostium and papilla that are segmented and rule sets then model a puncture path to the flank skin. Other anatomic points can be used from within the kidney to set a puncture path such as renal hilum, renal pelvis, general region of the kidney—posterior vs. Anterior, upper, mid or lower pole, general renal rotation, etc. Where a papilla is used as a datapoint to establish a puncture path, an optimal portion of the papilla 305 may be incorporated into the 2-point path proposals that are evaluated by the rule set. Probability rules will be applied to the modeled puncture path creating concentric cones of probability 415 passing through XYZ space 410 as an extrapolation from the minimum of two XYZ plane coordinates 405 that define the puncture path origin. A lower probability exists that the wire travels through a narrower central cone of projection 415a and a higher probability exists that the wire will travel through a wider cone of projection 415b. The further the wire travels, the wider the area of wire location probability exists 415c. Mapped onto the same XYZ map 410 is the flank window 420 which is defined by the flank window rule set. This will define the flank window as the space internal to a bordering of precious organs which cannot be in the puncture path. Some of these bordering organs include liver, spleen, colon, paraspinal muscle, pleura, ribs, anterior superior iliac spine (ASIS). The rule sets will define a margin of safety for the wire path projection to pass through this window and this will feed back to inform regions of the kidney selected for puncture and even regions of papilla 305 to achieve the correct angle of approach to pass safely through the window, given a number of other assumptions, for example, the cones of puncture path probability 415. Alternatively, a user can define the flank window based on their preference of distance to nearby precious organs and this field can be entered onto imaging data using a drawing tool or other resource. Finally, also mapped onto the XYZ grid 410 is the skin edge 430(a) and distance from the kidney to the skin 430(b). A rule set may be applied to ensure that too long a wire path 430(d) is avoided. For example, an obese patient's kidney-to-skin distance may be longer, thus a puncture angle 430(c) below the horizontal plane 430(b) will result in a longer wire path as the hypotenuse 430(d) of the triangle. This rule set may mandate a more horizontal wire path 430(e) in such an obese patient. An alternate exemplary embodiment applies this anatomic data field function and puncture path extrapolation calculation to either retrograde or antegrade renal punctures. If CT/fluoroscopy fusion is integrated, the 2-point puncture path origination calculation could be performed by 2D fluoroscopic position of the ureteroscope and for the third anterior/posterior position by modeling various points on the papilla 305 for puncture. Alternatively, EM navigation with 2-point sensor data in the ureteroscope can provide similar 2-point data for extrapolation. Data sets will consider confidence intervals in their calculations.

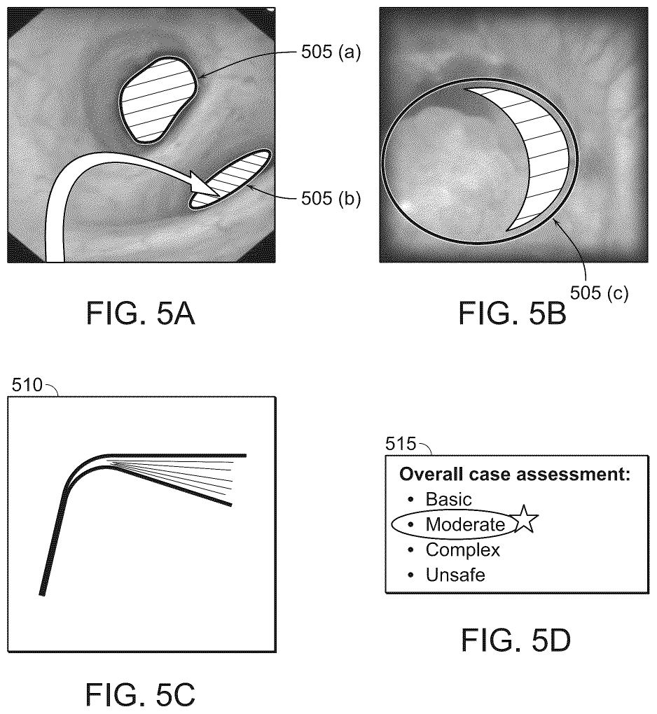

FIG. 5 demonstrates possible image guidance outputs 140. FIG. 505 shows image fusion of procedure proposal 130 and live video endoscopy 133. Regions to be avoided 505a and regions fit for puncture and arrow guidance thereto 505b are presented in a fused image. A plurality of papillae and their relative and or comparative suitability for puncture may be represented in the image output. FIG. 505c shows a face-on view of a renal papilla with a visual overlay guiding the surgeon to select a specific sub region of the papilla for puncture e.g. lateral aspect of papilla. FIG. 510 shows a calculated 2D image of a range of flexible ureteroscope angles that should be seen by the surgeon on 2D fluoroscopy in the operating room. This output is optional. Additional outputs may include an overall case rating 515 and image superimposition of video or CT/MRI 3D reconstruction or 2D representation onto fluoroscopy image data, or various alternate combinations of these. The image fusion function described herein can be applied to retrograde renal punctures or antegrade renal punctures, including those whose initial antegrade puncture of papilla is observed under direct ureteroscopic vision from a ureteroscope preplaced into a renal calyx.

In some embodiments, an electronic communications linkage can be established between the image guidance output and one or more of a camera, an item of robotic surgical equipment, a trocar, and an electrosurgery device. The electronic communications linkage can be configured for operating one or more of controls for robotic arms, trocars, electrosurgical devices, other surgical equipment and/or stereotactic video offering a three dimensional view of a surgical field.

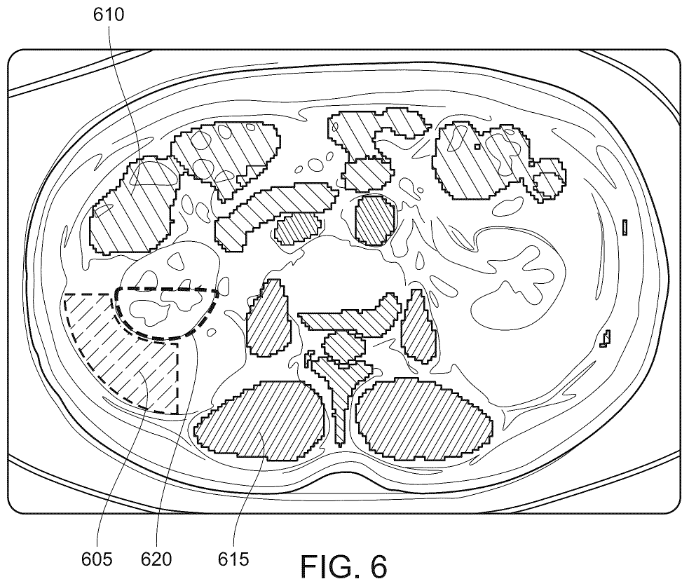

FIG. 6 is an example of a cross-sectional image with segmentation of relevant anatomic parts. The flank window 605 in this example can be segmented manually whereby a margin of safety to the precious perimeter organs is established by drawing the outer border of the flank window 605 some distance away from the precious perimeter organs such as bowel 610 or paraspinal muscle 615. Also shown is the posterior region 620 of the kidney segmented. This can provide orientation to posterior vs. anterior region of the kidney once in the reconstructed state. The posterior region of the kidney can be drawn (e.g. manually using a mouse and/or software drawing tool) to manually segment this region on each cross-sectional image or with learned AI modeling enhanced automatic segmentation of this region.

The learned AI modeling enhanced automatic segmentation comprises an application-specific integrated circuit (ASIC) for an artificial neural network connected to a communications network, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits configured to identify the coordinates of the window of safe passage.

The application-specific integrated circuit (ASIC) is used to make a prediction or classification of the coordinates of the window of safe passage based on some input data, which can be labeled or unlabeled. The algorithm will produce an estimate about a pattern in the data. An error function then evaluates the prediction of the model. If there are known examples, an error function can make a comparison to assess the accuracy of the model.

A model optimization process then occurs. If the model can fit better to the data points in the training set, then weights are adjusted to reduce the discrepancy between the known example and the model estimate. The algorithm will repeat this “evaluate and optimize” process, updating weights autonomously until a threshold of accuracy has been met. Supervised learning in particular uses a training set to teach models to yield the desired output. This training dataset includes inputs and correct outputs, which enables the model to learn over time. The algorithm measures its accuracy through the loss function, adjusting until the error has been sufficiently minimized.

As well, the coordinates of the window of safe passage are established not strictly by the position of the perimeter organs, but by the direct perimeter of the window of safe passage that has been drawn by person (manual segmentation e.g. applying a mouse and drawing tool onto the image) or software (AI assisted/software trained automatic segmentation) performing the segmentation of the perimeter of the window of safe passage. Other representations of this image reconstruction are weighting of visual prominence of various organ parts to optimize viewer understanding of a puncture path, virtual endoscopy, virtual reality, and a tool to permit zoom in, rotation of this image, virtual navigation through the image reconstructions. The sum of this will provide the surgeon a better 3-dimensional understanding of the surgical task along with its requirements, limitations, and challenges. Rule sets may be applied to calculate and present distance measurements from a proposed puncture path to nearby precious organs. Both retrograde and antegrade nephrostomy tract creation are considered exemplary embodiments of these applications.

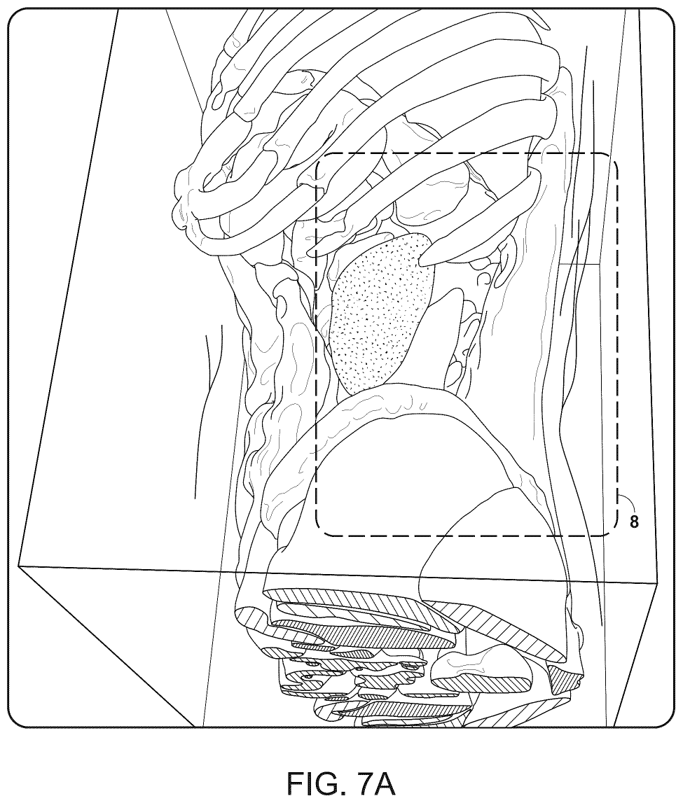

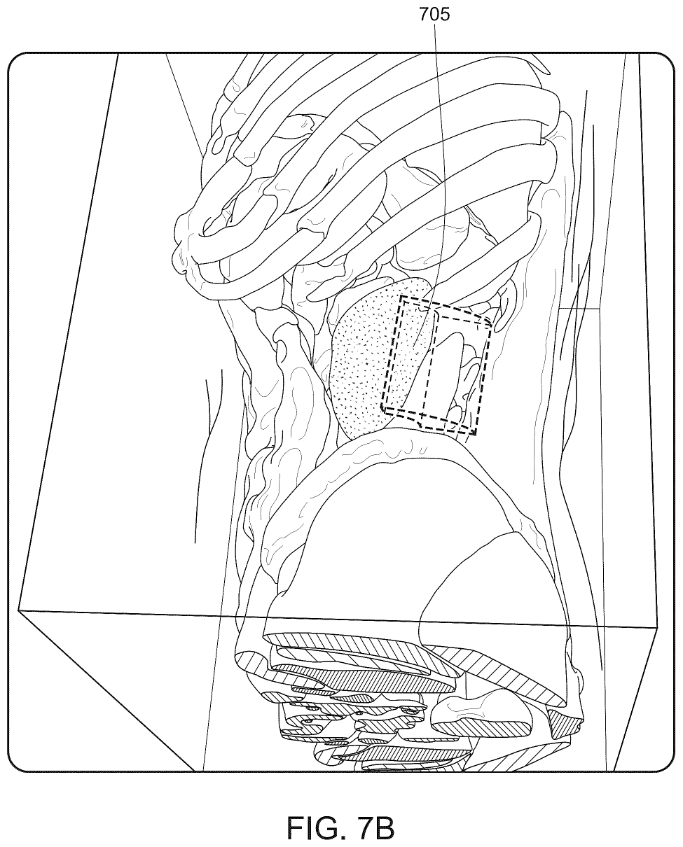

FIG. 7a is a 3D reconstruction of the image data segmentation performed in FIG. 6 without presentation of a segmented flank window. Modification of the segmented images of nearby organ images to clarify for the physician the kidney/skin/intervening organ relationships and puncture pathway, such as adding highlights, translucency to create a view most useful for and specific to a nephrostomy creation will be performed in a software post-processing environment. This can be done automatically based on image modification rule sets or manually by a viewer, or a combination of both. This process can be applied to an antegrade or retrograde nephrostomy puncture processes.

FIG. 7b is the same as FIG. 7 a above, with a highlight of the flank window 705 added. The flank window can be modeled using automatic segmentation or manual segmentation.

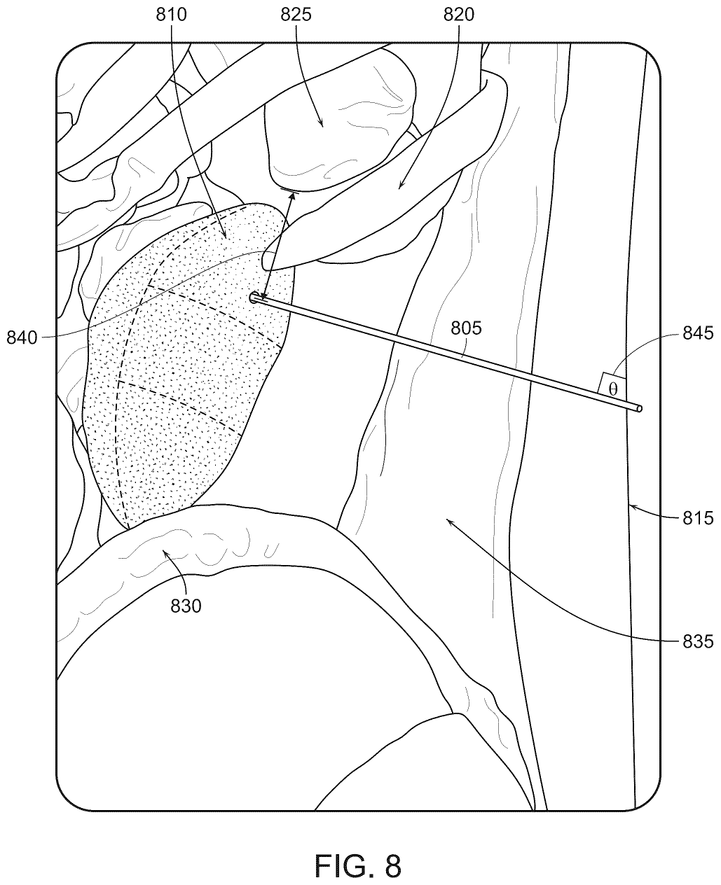

FIG. 8 demonstrates the output anatomic image reconstruction. The proposed puncture path 805 illustrated in a non-limiting fashion from a posterior upper pole region of the kidney 810 through the flank window to the flank skin 815 is visually presented in the context of regional anatomy such as the 12^{th} rib 820, spleen 825, anterior superior iliac spine 830 and paraspinal muscle 835. Kidney is divided into upper pole, mid pole and lower pole, anterior and posterior based on a segmentation process. In this figure the puncture travels through the posterior upper pole 810 although the puncture could traverse through one of many different locations in the kidney. This puncture path modeling can be a rule set output with or without input from the physician/user and may incorporate patient-specific rule set restrictions or physician/user selected puncture paths or specific rule sets selected by the physician/user. Alternatively, the physician/user can manually draw a puncture path via a computer software input capable drawing feature. Separate visual presentation of the flank window field on this image presentation is contemplated as on FIG. 7b but not visually represented visually here. Distance measurement from nearest point on puncture path to precious organs, for example the distance 840 from the puncture wire 805 to spleen 825 and angle of puncture path to skin 845 is calculated based on position of the modeled puncture line and segmented XYZ position data of precious organs. Various means of displaying measurement, angle or case summary data 515 to the physician/user are contemplated, such as color categories, highlighting of the organ most proximal to puncture, listing of measurement data, etc. 2D, 3D, virtual reality or virtual ‘travel through space’ presentations of data are contemplated. This process can be applied to antegrade or retrograde nephrostomy puncture processes.

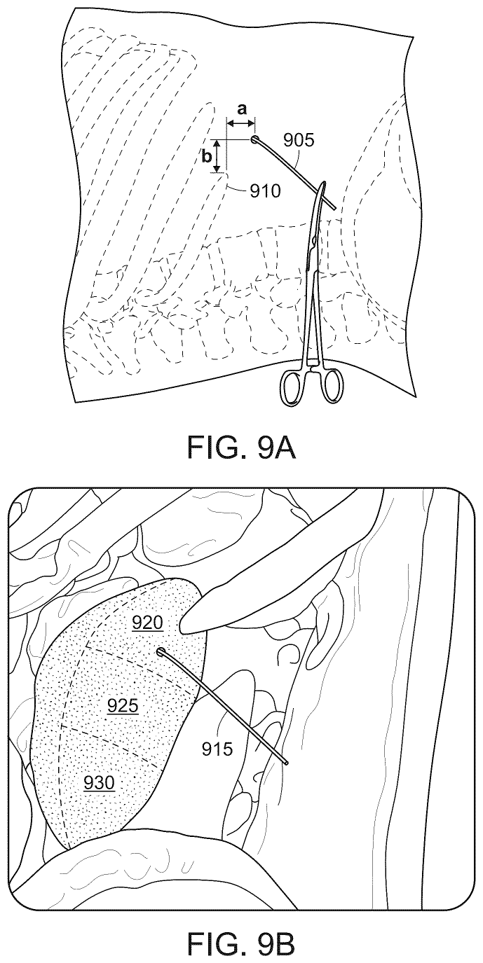

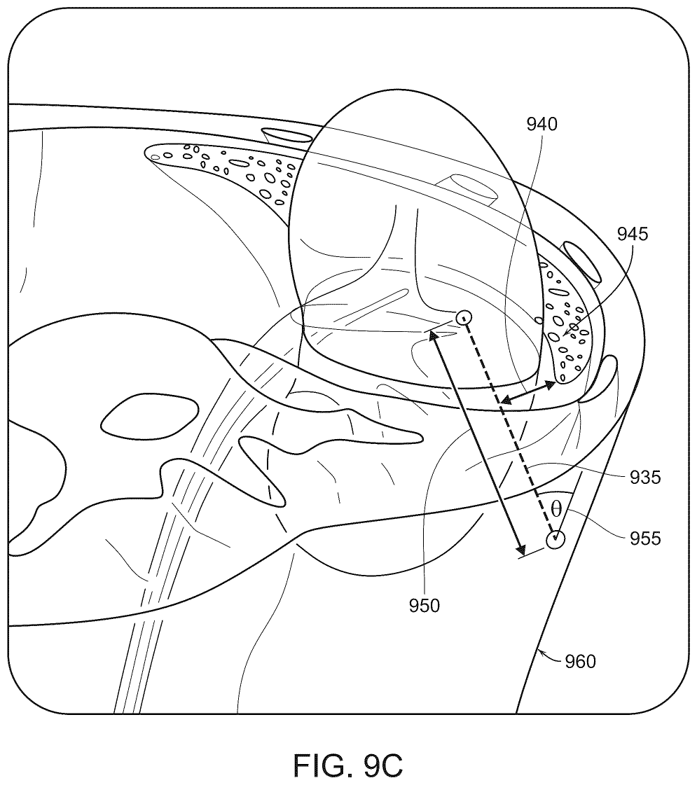

FIG. 9a demonstrates a created nephrostomy puncture emergent at the flank skin, with measurement referencing emergent puncture wire 905 whose position has a positional relation a and b in 2 planes to the tip of the 12th rib 910. Where the tip of the 12^{th} rib was previously segmented in the XYZ data field, this relative position data ‘a’ and ‘b’ is entered into a computer system using an input function along with data on renal puncture position, demonstrated in FIG. 9b. Together these two anatomic references are subjected to a rule set that interpolates a true puncture path that can be modeled onto reconstructed image of the patients flank anatomy along with presentation of calculated measurements to nearby precious organs. This process can be applied to a nephrostomy creation procedure performed in either an antegrade or retrograde puncture direction. FIG. 9b demonstrates a nephrostomy puncture 915 through the posterior upper pole of the kidney 920. Relevant preoperatively segmented regions of kidney shown here include posterior upper pole 920, posterior mid pole 925 and posterior lower pole 930. The image here models a non-limiting example of a puncture through the posterior upper pole of kidney 920 and is entered into a computer as an anatomic data point for the purpose of intraoperative modeling the created puncture path. Other preoperatively segmented renal landmarks can be used for this puncture landmark such as a specific calyx or a multiple of intrarenal anatomic data points. This process can be applied to a nephrostomy creation procedure performed in either an antegrade or retrograde puncture direction. FIG. 9c demonstrates modeling of a created nephrostomy puncture path 935 based on puncture data inputted as in FIG. 9a and FIG. 9b wherein an actual, created nephrostomy tract performed in either an antegrade or retrograde direction is modeled visually on the previously generated imagereconstruction of patient-specific flank anatomy. This example of an actual puncture path 935 is an interpolated line between the input data of puncture site at the flank skin and its relation to segmented identifiable anatomy e.g. 12^{th} rib tib 910 (FIG. 9a) and puncture site from the kidney (FIG. 9b). These two positional data points are inputted into a computer system where their XYZ position is modeled onto the previously established XYZ field of regional relevant organs. An interpolated line between these points and the anatomic relation of this interpolated line to previously segmented relevant organs on this same XYZ anatomic data field is thus established. The interpolated puncture line and its relation to surrounding organs along with relevant distances to nearby precious organs such as distance 940 from actual puncture path 935 to liver 945, distance of nephrostomy path 950 and angle of approach 955 of wire to skin 960, along with total puncture length 950 between kidney and skin 960 can thus be calculated and presented as a data output. The interpolated line can be modeled as simply the shortest line between the two points of XYZ anatomic position data input (skin and kidney). Additional modifying rule sets can be applied to interpolate the created puncture path, such as placing more weight on anterior/posterior distance from a reference point e.g. tip of 12th rib and less weight given to a data point such as cephalad/caudad distance from a reference point e.g. tip of 12th rib, etc. Rule sets to analyze the true puncture path can be applied to measure distance from a point on the puncture path to nearby precious organs, angle between puncture path to skin, determination of a procedure risk level such as 515, etc. Two-dimensional, three-dimensional, virtual reality or virtual ‘travel through space’ presentations of data are contemplated to aid the surgeon in understanding the nephrostomy puncture path they have created. These functions may be available in the operating room to aid in intraoperative decision making. This process can be applied to a nephrostomy creation procedure performed in either an antegrade or retrograde puncture direction.

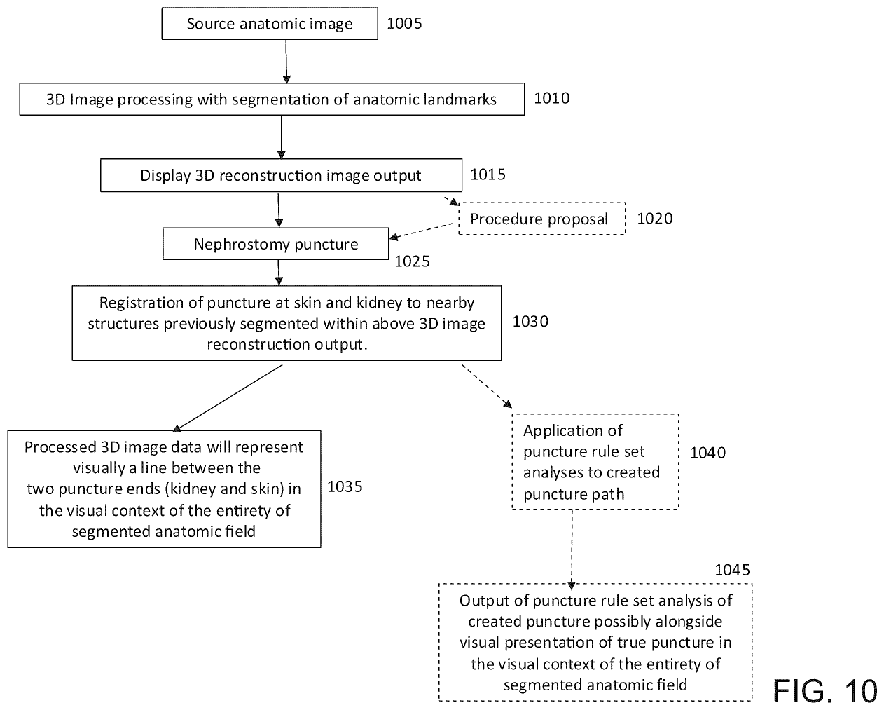

FIG. 10 is a flowchart depicting a process to represent a true created nephrostomy puncture path in the intraoperative setting prior to the physician committing to using the puncture path in an endourologic surgery. In exemplary embodiments, this process can be performed in a 2D or 3D image presentation environment although FIG. 10 in totality presents one exemplary embodiment of a 3D image reconstruction scenario. This represents a safety feature, to confirm a safe tract has been created before the nephrostomy puncture path is dilated to a larger diameter as part of a surgical procedure. For clarity, in the event of an unsafe puncture path, the puncture wire can be removed by the surgeon with little injury to the tissues from the very narrow puncture. The puncture can then be repeated with a corrected path or trajectory. A source image 1005 in this model is 3D processed with segmentation of anatomic landmarks 1010 in the preoperative setting These landmarks include soft tissue organs e.g. colon, muscle, liver, spleen, etc., as well as bony landmarks such as ribs, tip of ribs, etc. Included in segmented landmarks will be anatomic points that permit registration to intraoperative created puncture references by distance to said previously segmented anatomic reference points e.g. tip of 12th rib. The physician will review the 3D reconstruction image output. Puncture locations through skin and kidney are inputted into a computer 1030 to permit a puncture path to be represented on imaging. The input of these puncture points can be in reference to segmented nearby structures such as a non-limiting example of the tip of the 12th rib to permit accurate positioning of the line visually on a reconstructed 3D image set 1035 or additionally to permit application of further rule sets 1040 to evaluate 1045 the calculated puncture path's proximity to nearby precious organs, tract length, angles, puncture path analysis for safety etc. 515.

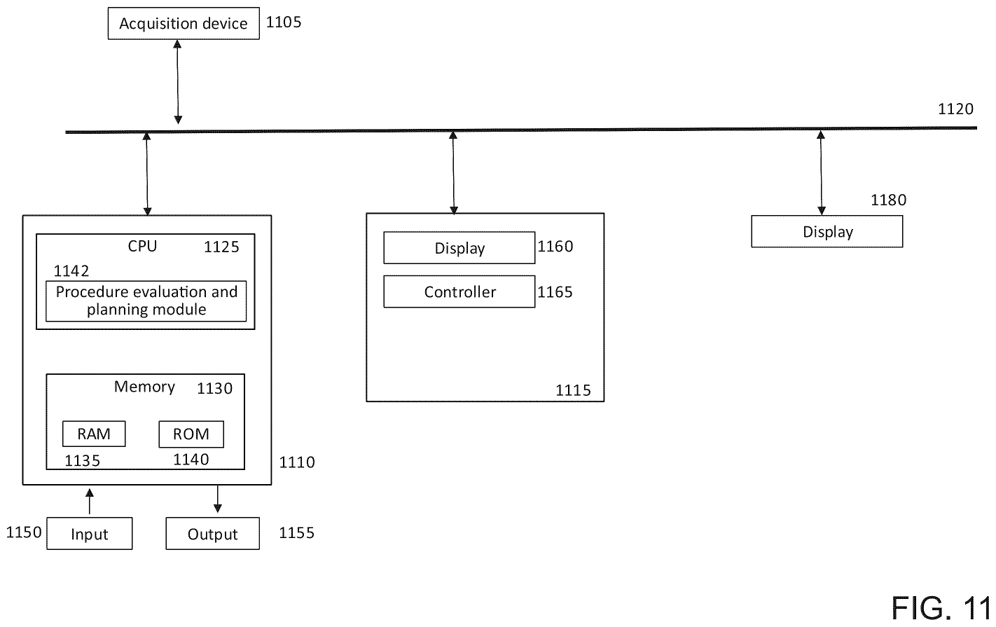

FIG. 11 is a block diagram illustrating a system for flexible ureteroscopic nephrostomy puncture procedure planning and intraoperative created puncture tract modeling according to an exemplary embodiment of this invention. As shown in FIG. 11, the system includes an acquisition device 1105, a computer 1110, an operator console 1115, and a display 1180 connected over a wired or wireless network 1120.

The acquisition device 1105 may be a computed tomography (CT) imaging device or any other three-dimensional (3D) high resolution imaging device such as magnetic resonance (MR) scanner. The acquisition device may additionally include a fluoroscopy image acquisition device such as a C-arm or fixed fluoroscopy table or image collected from CT scan scout/planning image which presents 2-dimensional x-ray data.

The computer 1110, which may be portable or laptop computer, includes a CPU 1125 and a memory 1130 connected to an input device 1150 and an output device 1155. The CPU 1125 includes an image reconstruction, evaluation and planning module 1142 which includes a nephrostomy line modeling function module which may contain one or more rule sets 1125 and methods. Although shown inside the CPU, any or all functions from among the image reconstruction, evaluation and planning module, and nephrostomy line modeling functions 1142 can be located outside the CPU 1125.

The memory 1130 includes a RAM 1135 and a ROM 1140. The memory 1130 can also include a database, disk drive, USB drive, Wi-Fi, Bluetooth, cloud connection, VPN, wired connection or combination thereof. The RAM 1135 functions as a data memory that stores data used during execution of a program in the CPU 1125 ad is used as a work area. The ROM 1140 functions as a program memory for storing a program executed in the CPU 1125. The input 1150 is constituted by a keyboard, mouse, etc. and the output 1155 is constituted by an LCD, CRT display, printer, etc.

The operation of the system in FIG. 11 can be controlled from the operator's console 1115, which includes a controller 1165, e.g. a keyboard or touchscreen, and a display 1160 which may have an integrated touch screen controller 1165. The operators console 1115 communicates with the computer 1110 and the acquisition device 1105 can be rendered by the computer 1110 and viewed on the display 1160. The computer 1110 can be configured to operate and display information provided by the acquisition device 1105 absent the operator's console 1115, by using e.g. the input 1150 and output 1155 devices to execute certain tasks performed by the controller 1165 and display 1160.

The operators console 1115 may permit manual anatomic inputs using text or a drawing tool onto an image represented on a screen and may further include any suitable image rendering system/tool/application that can process digital image data, including automatic anatomic segmentation, of an acquired image dataset (or portion thereof) to generate and display images on the display 1160. More specifically, the image rendering system may be an application that provides rendering and visualization of medical image data, and which executes on a general purpose or specific computer workstation. The computer 1110 can also include the above-mentioned image rendering system/tool/application.

It is considered that some or more of the above functions may be remotely located within the same building or offsite at a remote location with connection via secure data transfer over VPN, cloud, Wi-Fi, Bluetooth, or other modalities. Functions which may be hosted offsite may include the CPU 1125 and the memory components 1130.

Visual outputs such as puncture assessment 515, puncture path image modeling onto 2D or 3D image reconstructions, measurement and angle calculations are displayed on monitor 280.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specifications and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A method of defining a safe pathway for renal nephrostomy, comprising:
obtaining an imaging reconstruction of previously acquired source patient-specific imaging data demonstrating the kidney and other structures in the region of the path between the kidney and the flank skin;
segmenting a plurality of anatomic reference points comprising one or more of any number of renal anatomy points selected from the group comprising posterior kidney field, upper pole, lower pole, mid pole, calyces, infundibulae, papillae, calyceal fornices, renal hilum), the position and terminal points of one or more ipsilateral ribs, colon, pleura, liver, spleen, paraspinal muscle, pararenal and perirenal fat, fascia, flank musculature, skin, and subcutaneous fat;
displaying a visual image reconstruction presenting selected anatomic elements onto a single visual spatial model; and
representing an anatomical passage free of precious organs comprising one or more of liver, spleen, bowel, intestine, pleura, rib, and paraspinal muscle between a patient kidney and nearby flank skin as an anatomical region on a two-dimensional (2D) or 3D model.

2. The method of claim 1, further comprising drawing a path onto the model between a site at the skin and a site at the kidney representing a puncture path between flank skin and kidney to evaluate a relationship between the puncture path and nearby relevant anatomical structures.

3. The method of claim 1, wherein images of specific anatomic regions or organs in the visual image reconstruction presenting selected anatomic elements onto a single visual spatial model are modified for the purpose of enhancing perception of the path between kidney and flank skin while still representing relevant nearby organs.

4. The method of claim 1, wherein a path is modeled by selecting one puncture site at the skin and one site at the kidney and interpolating a line between these points.

5. The method of claim 4, wherein the position of the skin puncture site is modeled in planar relation to a nearby bony landmark, such as the tip of a rib.

6. The method of claim 1, wherein the anatomical passage can be quantified or categorized by linear measurements between nearby organs (e.g. space between liver and paraspinal muscle) to describe the puncture path.

7. The method of claim 1, wherein renal specific anatomic features comprising one or more of renal rotation, anterior vs. posterior regions of kidney, upper/mid/lower pole regions of kidney, renal infundibulae, renal papillae, subsegments of renal papillae, and any renal masses are incorporated in the visual image to enhance position and structure information.

8. The method of claim 1, further comprising applying translucency in the image reconstruction to various anatomic elements to permit enhanced visual understanding of the path between the kidney and the flank skin.

9. The method of claim 1, wherein the previously acquired source patient-specific imaging data comprises one or more of CT urography and/or retrograde pyelography.

10. The method of claim 2, wherein the puncture path passes through perirenal and pararenal fat and flank fascia as it traverses between kidney and flank skin.

11. The method of claim 2, further comprising formatting and processing the visual image reconstruction to permit ‘drive through’ or ‘virtual puncture’ journey in either direction between kidney and flank skin in 3D space using a virtual reality or alternative software feature.

12. The method of claim 2, wherein the puncture path is overlaid onto the previously acquired source patient-specific imaging data.

13. The method of claim 1, further comprising establishing an electronic communications linkage with one or more of a camera, an item of robotic surgical equipment, a trocar, and an electrosurgery device.

14. The method of claim 13, further comprising operating one or more of controls for robotic arms and/or stereotactic video offering a three-dimensional view of a surgical field.

15. The method of claim 2, wherein the path is used to inform a retrograde nephrostomy creation procedure.

16. The method of claim 2, wherein the path is used to inform an antegrade nephrostomy creation procedure.

17. The method of claim 2, wherein anatomic points referencing a created puncture tract are inputted to the preoperatively prepared multiplanar anatomic field data and a puncture path is interpolated between these datapoints and presented to the physician during a procedure.

18. The method of claim 17, wherein a distance from a point on the interpolated puncture path to a nearby organ is presented to a physician during a procedure.

19. The method of claim 2, further comprising presenting an image tracking function wherein an image fusion output is presented between live video endoscopy and 3D reconstructed patient-specific anatomy to provide live positional information for a nephrostomy creation procedure.

20. The method of claim 19, wherein the image tracking function is added to a live intraoperative navigation updated one or more times over a regular or irregular interval.

21. The method of claim 16, wherein the image fusion output is between live video endoscopy and a preoperatively planned puncture path modeled inside a 3D anatomic field reconstruction.

22. An application-specific integrated circuit (ASIC) for an artificial neural network, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits configured to analyze an imaging reconstruction of previously acquired source patient-specific imaging data demonstrating the kidney and other structures in the region of the path between the kidney and the flank skin.

23. The application-specific integrated circuit (ASIC) of claim 22, wherein the plurality of synaptic circuits are further configured to segment a plurality of anatomic reference points comprising one or more of any number of renal anatomy points (e.g. posterior kidney field, upper pole, lower pole, mid pole, calyces, infundibulae, papillae, calyceal fornices, renal hilum), the position and terminal points of one or more ipsilateral ribs, colon, pleura, liver, spleen, paraspinal muscle, pararenal and perirenal fat, fascia, flank musculature, skin, subcutaneous fat.

24. The application-specific integrated circuit (ASIC) of claim 22, wherein the plurality of synaptic circuits are further configured to calculate an anatomical passage free of precious organs comprising one or more of liver, spleen, bowel, intestine, pleura, ribs, and paraspinal muscle between a position within a patient kidney and a position on nearby flank skin.

25. The application-specific integrated circuit (ASIC) of claim 22, wherein the application-specific integrated circuit (ASIC) is connected to a communications network and configured to output a visual image reconstruction comprising a two-dimensional (2D) or 3D model.

26. The application-specific integrated circuit (ASIC) of claim 22, wherein the plurality of synaptic circuits are further configured to first receive intraoperative anatomic data points representing portions of a created nephrostomy puncture, second to interpolate a puncture path line between these inputted anatomic data points, and third to present this puncture path in context of surrounding anatomy.

27. The application-specific integrated circuit (ASIC) of claim 22, wherein the plurality of synaptic circuits are further configured to utilize the array to calculate distance from a point on the puncture path to nearby surrounding organs and/or propose a puncture path between kidney and flank skin.

28. A system, comprising:
A processor;
A computer-readable memory device;
And a display device, wherein the processor is configured for:
obtaining an imaging reconstruction of previously acquired source patient-specific imaging data demonstrating the kidney and other structures in the region of the path between the kidney and the flank skin stored on the computer-readable memory device;
segmenting a plurality of anatomic reference points comprising one or more of any number of renal anatomy points (e.g. posterior kidney field, upper pole, lower pole, mid pole, calyces, infundibulae, papillae, calyceal fornices, renal hilum), the position and terminal points of one or more ipsilateral ribs, colon, pleura, liver, spleen, paraspinal muscle, pararenal and perirenal fat, fascia, flank musculature, subcutaneous fat, skin;
displaying a visual image reconstruction presenting selected anatomic elements onto a single visual spatial model; and
representing an anatomical passage free of precious organs comprising one or more of liver, spleen, bowel, intestine, pleura, ribs, and paraspinal muscle between a patient kidney and nearby flank skin as an anatomical region on a two-dimensional (2D) or 3D model on the display device.

29. The system of claim 28, further comprising one or more imaging devices communicatively coupled to the processor and configured for acquired source patient-specific imaging data demonstrating the kidney and other structures in the region of the path between the kidney and the flank skin.

30. The system of claim 28, wherein one or more known anatomic puncture locations are added to a computer system to interpolate a puncture path and represent the created puncture in relation to the patient-specific regional anatomic field.

31. The system of claim 28, wherein the system first receives intraoperative anatomic data points representing portions of a create nephrostomy puncture, second applies rule sets to interpolate a puncture path line between these inputted anatomic data points, and third to present this puncture path in context of surrounding anatomy.
